(19) United States
(12) Patent Application Publication
(10) Pub. No.: US 2008/0096923 A1
(43) Pub. Date: Apr. 24, 2008

(54) Methods For Diagnosing And Treating Diabetic Microvascular Complications

(76) Inventor: Aniz Girach, Hampshire (GB)

Correspondence Address: ELI LILLY & COMPANY, PATENT DIVISION, P.O. BOX 6288, INDIANAPOLIS, IN, 46206-6288, US

(21) Appl. No.: 11/572,228

(22) PCT Filed: Jul. 13, 2005
(86) PCT No.: PCT/US05/24908

§ 371 Date: Jan. 17, 2007

Related U.S. Application Data
(60) Provisional application No. 60/590,497, filed on Jul. 23, 2004.
(60) Provisional application No. 60/637,528, filed on Dec. 20, 2004.

(51) Int. Cl. A61K 31/4545; A61K 31/407; A61K 31/4184; A61K 31/4188; A61P 25/02; A61P 9/00; A61P 13/12; A61K 31/428; A61K 31/4412

(52) U.S. Cl. 514/318; 514/351; 514/367; 514/381; 514/389; 514/410

(57) ABSTRACT

The present invention relates to a method for treating one or more diabetic microvascular complications in a patient in need of said treatment comprising: (a) diagnosing the severity of at least three different microvascular complications in said patient by calculating a diabetes microvascular complications score with a diabetes microvascular complications scoring tool; and (b) administering to said patient in need thereof a therapeutic amount of a compound selected from the group consisting of ruboxistaurin, enzastaurin, PKC 412, candesartan cilexetil, fidarestat, lidorestat, pyridoxamine and pegaptanib, or a pharmaceutically acceptable salt thereof, and ranibizumab; in an amount that is effective in treating one or more diabetic microvascular complications in said diabetic patient.

### BACKGROUND OF THE INVENTION

Diabetes mellitus is a global health problem, affecting all age groups. Currently, around 177 million people have diabetes worldwide; however, the World Health Organization (WHO) project that this number will increase to at least 300 million by 2025. The diabetes epidemic relates in particular to Type 2 diabetes, which accounts for around 90% of all diabetes cases. The increased prevalence of Type 2 diabetes can be attributed to the aging population and rising incidence of obesity in the developed countries, among other factors.

Prevention of complications specific to diabetes is a key issue because of the morbidity and mortality associated with the disease. Clinically significant morbidity may often develop before diagnosis. Between one-third and one-half of all people with diabetes have evidence of organ or tissue damage. Although not everyone with diabetes will develop a complication, a recent epidemiological study reported that two or more complications are apparent in almost one fifth of people with diabetes. Morgan C L, Currie C J, Stott N C H, et al.; “The prevalence of multiple diabetes-related complications.” Diabet Med 17:146-151 (2000).

If diabetes is undetected or not treated, or if its complications are poorly managed it can have a devastating impact on quality of life. Diabetes also places a significant burden on health care costs, which consist of direct costs of medical and community care, and indirect costs such as unemployment and premature mortality. The major single item of diabetes expenditure is hospital admissions for the treatment of complications, with direct health care costs ranging from 2.5% to 15% of annual health care budgets. Indirect costs, including loss of productivity, may be as much as five times the direct health care cost.

Landmark studies, including the Diabetes Control and Complications Trial (DCCT) and the United Kingdom Prospective Diabetes Study group (UKPDS) have shown that intensive control of blood glucose levels and tight blood pressure control reduces the risk of complications related to diabetes. The Diabetes Control and Complications Trial Research Group; “The effect of intensive treatment of diabetes on the development and progression of long-term complications in insulin-dependent diabetes mellitus;” N. Eng. J. Med 329:977-986 (1993); Turner R, Holman R, Stratton I, et al.; “Tight blood pressure control and risk of macrovascular and microvascular complications in type 2 diabetes: UKPDS 38;” BMJ 317:703-713 (1998). In addition, early identification of risk factors can help reduce the development and progression of microvascular complications, and improve patients' quality of life. Although chronic hyperglycaemia is the most powerful risk factor for the development and progression of diabetic complications, disease duration, hypertension, age at onset, smoking, age, sex and genetic factors may all play a part.

Microvascular complications develop in most people with Type 1 and Type 2 diabetes and are associated with clinically significant morbidity and mortality. It has been suggested that subsets of patients with Type 1 diabetes may have a genetically determined susceptibility to microvascular complications as not all people with Type 1 diabetes and very high blood glucose levels develop complications. Conversely, some develop complications even if blood glucose levels are only slightly elevated. Type 2 diabetes is increasing across all ethnic groups, particularly among black and minority groups. Because Type 2 diabetes is often not diagnosed until the patient has had the disease for many years, long-term complications may be present at the time diabetes is discovered.

Although there are several known risk factors, chronic hyperglycaemia is a major initiator of diabetic retinopathy, nephropathy and neuropathy. The DCCT has shown that the more time individuals are exposed to chronically elevated plasma glucose levels, the greater their risk of microvascular complications. In addition, the deleterious effects of hyperglycaemia on the microcirculation have been shown to persist for a considerable time after glucose levels have decreased. Both the DCCT and UKPDS have shown that intensive glycaemic management slows the progression of microvascular complications in Type 1 and Type 2 diabetes, and thereby improves quality of life. Although intensive therapy may adversely affect the development of retinopathy, the DCCT concluded that the long-term benefits of intensive insulin therapy greatly outweigh the early risks of retinopathy. The Diabetes Control and Complications Trial Research Group. “Early worsening of diabetic retinopathy in the Diabetes Control and Complications Trial.” Arch. Ophthalmol. 116:874-886 (1998).

Tight control of blood pressure in both hypertensive and normotensive patients with Type 2 diabetes has also been shown to reduce the risk of the development and progression of microvascular complications. Turner R, Holman R, Stratton I, et al. “Tight blood pressure control and risk of macrovascular and microvascular complications in type 2 diabetes: UKPDS 38.” BMJ 317:703-713 (1998). Data from a recent cost-analysis study show that the policies to improve control of blood glucose and blood pressure of people with Type 2 diabetes are not only effective in reducing complications associated with the disease, but are also cost-effective. Gray A, Clarke P, Farmer A et al, for the United Kingdom Propsective Diabetes Study (UKPDS) Group. “Implementing intensive control of blood glucose concentration and blood pressure in type 2 diabetes in England: cost analysis (UKPDS 63).” BMJ 325:860 (2002).

A wide range of other risk factors have also been investigated including age at onset, smoking, height, age, genetic factors, duration of diabetes, and unfavorable lipid profiles. The levels of AGE in patients with both retinopathy and nephropathy have been shown to be elevated, but whether this is a reflection of the presence of poor glycaemic control in these patients or whether the AGEs are an independent risk factor, is not clear. The duration of diabetes is also a factor that is clearly involved in the prevalence of the complications. However, it is not known if the prevalence of the complications increases because of cumulative microvascular damage related to the duration, or whether it is a reflection of the escalating involvement of other risk factors such as poor glycaemic control and hypertension.

Along with the presence of external risk factors, some associations have been noted between complications themselves. The DCCT reported that even very early in the development of the complications there is a relationship between retinopathy and nephropathy. Molitch M E, Steffes M W, Cleary P A, et al. “Baseline analysis of renal function in the Diabetes Control and Complications Trial. The Diabetes Control and Complications Trial Research Group.” Kidney Int 43:668-674 (1993). Within the study group, which had evidence of minimal retinopathy at baseline, 10% had elevated urinary albumin excretion rate levels. There was also a strong relationship between elevated urinary albumin excretion rate levels and more advanced degrees of diabetic retinopathy. Rossing, et al. also reported the presence of retinopathy in patients with Type 1 diabetes to be predictive of onset of microalbuminuria, although no difference was found between background or proliferative retinopathy. Rossing P, Hougaard P, Parving H H. “Risk factors for development of incipient and overt diabetic nephropathy in type 1 diabetic patients: a 10-year prospective observational study.” Diabetes Care 25(5):859-864 (2002). The EURODIAB study showed that the correlation between increasing blood pressure and albumin excretion rate was only confirmed in patients who also had retinopathy, independently of glycaemic control or diabetes duration, suggesting that diabetic retinopathy, in association with increased blood pressure, is an important independent risk factor for nephropathy progression. Stephenson J M, Fuller J H, Viberti G C et al, the EURODIAB IDDM Complications Study Group. “Blood pressure, retinopathy and urinary albumin excretion in IDDM: the EURODIAB IDDM Complications Study.” Diabetologia 38:599-603 (1995). On the other hand, the fact that diabetic retinopathy and nephropathy can occur in isolation suggests there are important differences in some aspects of the pathogenesis of these two diabetic microvascular complications.

Ongoing research has led to a better understanding about diabetes and its related complications. Although currently available data on the evolution of long-term complications are limited, the EDIC study should provide important evidence of micro- and macrovascular endpoints and definitive data on Type 1 diabetes as distinct from Type 2 diabetes. Clinical trials are also currently in progress looking at a number of approaches to designing treatments to prevent the adverse effects of hyperglycaemia including aldose reductase inhibitors, AGE inhibitors and inhibitors of protein kinase C (PKC).

Despite good long-term glycaemic and blood pressure control, diabetes remains a major cause of blindness, renal failure and amputations, all of which result in significant health care expenditure. As the incidence of diabetes continues to rise, the burden of microvascular complications will increase in the future. To further reduce the associated morbidity and mortality it is essential that factors associated with the onset and progression of diabetes-related complications are identified as early as possible. In addition, risk factors, such as smoking and hypercholesterolaemia need to be modified and new interventions developed to tackle unmodifiable risk factors, such as disease duration and genetics. In this respect, considering all three complications as inter-related and the need to enquire proactively about complications that may not be present or apparent yet, but could develop or become more apparent in the future, may well facilitate early detection of microvascular disease. This invention represents a unifying method of assessing the overall level of microvascular activity and an improved method for determining patients in need of treatment for diabetic microvascular complications. This invention represents a significant step towards the early identification of those patients at risk of the microvascular complications of diabetes.

### BRIEF SUMMARY OF THE INVENTION

The present invention relates to a method for treating one or more diabetic microvascular complications in a patient in need of said treatment comprising:

(a) diagnosing the severity of at least three different microvascular complications in said patient by calculating a diabetes microvascular complications score with a diabetes microvascular complications scoring tool; and

(b) administering to said patient in need thereof a therapeutic amount of a compound selected from the group consisting of ruboxistaurin, enzastaurin, PKC 412, candesartan cilexetil, fidarestat, lidorestat, pyridoxamine and pegaptanib, or a pharmaceutically acceptable salt thereof, and ranibizumab; in an amount that is effective in treating one or more diabetic microvascular complications in said diabetic patient.

### DETAILED DESCRIPTION OF THE INVENTION

As used herein, the term “diabetic microvascular complications” refers to any complication of diabetes mellitus that is wholly or in part due to a microvascular mediated cause which includes (but is not limited to): diabetic eye disease (including retinopathy, macular edema, blindness), diabetic nerve disease (including neuropathy, autonomic neuropathy, foot ulceration, amputation), and diabetic kidney disease (including microalbuminuria, proteinuria, nephropathy, end-stage renal disease, hemodialysis).

As used herein, the term “compound of the invention” refers to ruboxistaurin, enzastaurin, PKC 412, candesartan cilexetil, fidarestat, lidorestat, pyridoxamine, pegaptanib, a pharmaceutically acceptable salt thereof, or ranibizumab.

Diabetic retinopathy is a major sight-threatening complication and a leading cause of visual disability and blindness. It can involve the peripheral retina, the macula, or both. The range of severity includes background (mild to moderate non-proliferative), preproliferative (severe and very severe non-proliferative), proliferative, and advanced retinopathy. Harding S.; Extracts from “Concise Clinical Evidence. Diabetic retinopathy. BMJ; 326:1023-1025 (2002). The impact of retinopathy with its associated clinical features is fundamentally similar in Type 1 and Type 2 diabetes. Cunha-Vaz J.; “Lowering the risk of visual impairment and blindness.” Diabet Med. 15 (Suppl. 4):S47-S50 (1998); Schmechel H, Heinrich U.; “Retinopathy and nephropathy in 772 insulin-treated diabetic patients in relation to the type of diabetes.” Diabetes & Metabolisme 19:138-142 (1993).

The prevalence of diabetic retinopathy varies widely depending on the population studied. Background retinopathy however, is almost universal after 20 years of diabetes, while proliferative diabetic retinopathy affects 70% of people with Type 1 diabetes after 30 years duration. Orchard T J, Dorman J S, Maser R E, et al.; “Prevalence of complications in IDDM by sex and duration. Pittsburgh Epidemiology of Diabetes Complications Study II.” Diabetes 39:1116-1124 (1990).

Incidence data from the Wisconsin Epidemiologic Study of Diabetic Retinopathy (WESDR) showed that insulin-taking people diagnosed to have diabetes before the age of 30 years have the highest prevalence of diabetic retinopathy (71%), four-year incidence (59%) and progression to proliferative diabetic retinopathy (11%), while older-onset people diagnosed to have diabetes at or after 30 years of age and not taking insulin had the lowest prevalence (39%), incidence (34%) and progression to proliferative diabetic retinopathy (3%). Klein R, Klein B E K, Moss S E; “The Wisconsin Epidemiologic Study of Diabetic Retinopathy: an update.” Aust N Z J Ophthalmol 18:19-22 (1990).

A number of risk factors have been shown to determine the development and progression of diabetic retinal disease, including duration of diabetes, poor glycaemic control, raised blood pressure, age at onset, increasing number of microaneurysyms, microalbuminuria and proteinuria, and pregnancy.

Epidemiological studies have established that the prevalence of retinopathy increases with duration of diabetes and age at onset. Although background retinopathy may be present in some cases of recently diagnosed patients with Type 1 or Type 2 diabetes, the prevalence of retinopathy is significantly higher in patients with long-standing disease. Straub, et al. found a significant correlation with disease duration and retinopathy when plotting prevalence versus duration of diabetes in a study collecting data from patients with both Type 1 and Type 2 diabetes over a period of five years. Straub R H, Zietz B, Palitzsch, et al.; “Impact of disease duration on cardiovascular and papillary autonomic nervous function in IDDM and NIDDM patients.” Diabetes Care 19:960-967 (1996). The Pittsburgh Epidemiology of Diabetes Complications Study, which evaluated 657 patients with childhood-onset (<17 years) Type 1 diabetes, found that almost all patients had background retinopathy in both age groups by 14 years of diabetes. After 25-29 years of diabetes, three quarters of patients aged 18-29 years and more than half of patients aged 30 years or over had proliferative retinopathy.

Wirta, et al. also reported that all forms of diabetic retinopathy were frequent in patients with longer diabetes duration (5-25 years). In patients with recently diagnosed Type 2 diabetes, the prevalence for non-specific, background and proliferative retinopathies were 17%, 6% and 0% respectively. However, in patients with long-term diabetes, the prevalence increased to 40%, 31% and 8%. Wirta O R, Pasternack A I, Oksa H H, et al.; “Occurrence of late specific complications in type II (non-insulin dependent) diabetes mellitus.” Journal of Diabetes and Its Complications 9:177-185 (1995). Similar findings were reported in the WESDR, in which the prevalence of proliferative diabetic retinopathy was more prevalent among patients with prolonged duration of disease (Table 1). Klein R K, Klein B E K, Moss S E, et al.; “The Wisconsin Epidemiologic Study of Diabetic Retinopathy. II. Prevalence and risk of diabetic retinopathy when age at diagnosis is less than 30 years.” Arch. Ophthalmol. 102:520-526 (1984).

TABLE 1
Prevalence of proliferative diabetic retinopathy in the Wisconsin
Epidemiologic Study of Diabetic Retinopathy

| | Prevalence of diabetic |
|---|---|
| Duration of diabetes | proliferative retinopathy |
| Less than 5 years | 0% |
| 10 years | 4% |
| 15 years | 25% |
| 35 years | 67% |

Schmechel, et al. found that insulin-treated patients with Type 1 and Type 2 diabetes do not differ in their frequency of retinopathy given identical duration of disease. Retinopathy was diagnosed in 238 (52.5%) patients with Type 1 diabetes and in 534 (50.3%) insulin-treated patients with Type 2 diabetes. Duration of diabetes was longer in patients with retinopathy, both in Type 1 diabetes (18 vs. 8 years) and in Type 2 diabetes (16 vs. 12 years). After an average duration of the disease of 14 years, no differences were reported in the degree of retinopathy between the two groups. Schmechel H, Heinrich U.; “Retinopathy and nephropathy in 772 insulin-treated diabetic patients in relation to the type of diabetes.” Diabetes & Metabolisme 19:138-142 (1993).

Other studies have reported frequencies of microvascular complications in relation to age at onset of diabetes. Henricsson, et al. showed that the prevalence of retinopathy in adult patients with Type 1 diabetes (mean duration 20.8 years) was significantly greater in those diagnosed during puberty compared with those diagnosed before puberty. Henricsson M, Nilsson A, Janzon L, et al.; “The effect of glycaemic control and the introduction of insulin therapy on retinopathy in non-insulin-dependent diabetes mellitus.” Diabet. Med. 14:123-131 (1997). Similar results were reported by Vogt et al, whereby there was a significantly increased risk of diabetic complications such as retinopathy in patients with diabetes onset in puberty (up to diabetes duration of 20 years), compared to diabetes onset before puberty. Vogt L, Jutzi E, Michaelis D.; “Different frequencies of diabetic complications in insulin-treated patients with diabetes of comparable duration, in relation to age at onset of diabetes.” Soz. Praventivmed. 37(5):231-236 (1992).

Applicants theorize that patients developing diabetes before the onset of puberty are protected for some time against the complications, but the overall risk of developing retinopathy remains the same, as after 20 years duration, no significant differences between the groups were reported. Kostraba, et al. reported however, that the effect of duration on diabetes complications is not consistent. Kostraba J N, Dorman J S, Orchard T J, et al.; “Contribution of diabetes duration before puberty to development of microvascular complications in IDDM subjects.” Diabetes Care 12:686-693 (1989). This study, which evaluated three diverse populations of adolescent and adult patients with Type 1 diabetes found that overall, the prevalence of retinopathy in 552 adult patients was significantly greater in patients diagnosed during puberty compared with those diagnosed before puberty. Similar analyses by postpubertal duration showed no difference between the two groups. The findings did not appear to be due to a confounding effect of age. The same trend for the prevalence of retinopathy was also found in additional analyses of 239 patients with a mean duration of diabetes of 8.3 years.

Microaneurysms are the hallmark of diabetic retinopathy, and are the earliest clinically recognisable features of the condition. The UKPDS found that the presence of microaneurysms alone and also the number of microaneurysms in patients with Type 2 diabetes who had either no retinopathy or microaneurysms only at entry had a highly predictive value for worsening of retinopathy at 3, 6, 9 and 12 years after entry in the study. Kohner E M, Stratton I M, Aldington S J, et al., for the UK Prospective Diabetes Study (UKPDS) Group. “Microaneurysms in the development of diabetic retinopathy (UKPDS 42).” Diabetologia 42:1107-1112 (1999). The UKPDS also looked at the relationship between the severity of retinopathy and progression to photocoagulation in 3,709 patients with Type 2 diabetes. Kohner E M, Stratton I M, Aldington S J, et al.; “Relationship between the severity of retinopathy and progression to photocoagulation in patients with Type 2 diabetes mellitus in the UKPDS (UKPDS 52).” Diabet. Med. 18(3):178-184 (2001). Severity was categorised as no retinopathy, microaneurysms in one eye only, and microaneurysms in both eyes or more severe retinopathy features. Results showed that few patients without retinopathy progress to photocoagulation in the following 3 to 6 years. However, 15.3% patients with more severe retinopathy lesions required photocoagulation by 3 years and 31.9% by 9 years (Table 2).

TABLE 2
Progression to photocoagulation

| | Progression to photocoagulation | | |
|---|---|---|---|
| At baseline | 3 years | 6 years | 9 years |
| No retinopathy | 0.2% | 1.1% | 2.6% |
| (n = 2,316) | | | |
| Microanuerysms in | 0% | 1.9% | 4.7% |
| one eye only | | | |
| (n = 708) | | | |
| More severe | 15.3% | 25.2% | 31.9% |
| retinopathy | | | |
| features (n = 509) | | | |

Several studies have demonstrated that elevated plasma glucose levels correlate with microvascular complications in Type 1 diabetes. Chase, et al. reported that patients with Type 1 diabetes with poor long-term glycaemic control had 2.5 times the prevalence of more severe retinal involvement than that found in subjects with good long-term control. Chase H P, Jackson W E, Hoops S L, et al.; “Glucose control and the renal and retinal complications of insulin-dependent diabetes.” JAMA 261:1155-1160 (1989). The DCCT, which was designed to study the effect of enforcing optimised metabolic control on the complications of diabetes in a large cohort of 1,441 patients aged 13 to 39 years with Type 1 diabetes for 1 to 15 years, found that intensive therapy effectively delayed the onset and slowed the progression of diabetic retinopathy. In patients with no retinopathy at baseline, intensive therapy reduced the risk of developing retinopathy by 76% as compared with conventional therapy. In patients with mild retinopathy, intensive therapy reduced the development of proliferative or severe non-proliferative retinopathy by 47%. The Diabetes Control and Complications Trial Research Group. “The effect of intensive treatment of diabetes on the development and progression of long-term complications in insulin-dependent diabetes mellitus.” N. Eng. J. Med. 329:977-986 (1993).

Poor glycaemic control is significantly associated with retinopathy progression also in patients with Type 2 diabetes. Henricsson M, Nilsson A, Janzon L, et al.; “The effect of glycaemic control and the introduction of insulin therapy on retinopathy in non-insulin-dependent diabetes mellitus.” Diabet. Med. 14:123-131 (1997). Data from the UKPDS demonstrated that intensive blood glucose control after diagnosis of Type 2 diabetes prevents the complications of diabetes and reduces associated morbidity and mortality. United Kingdom Prospective Diabetes Study (UKPDS) Group. “Intensive blood glucose control with sulphonylureas or insulin compared with conventional treatment and risk of complications in patients with type 2 diabetes (UKPDS 33).” Lancet 352:837-853 (1998). Most of the risk reduction in the diabetes-related aggregate endpoint was due to a 25% risk reduction in microvascular endpoints, including the need for retinal photocoagulation. Conversely, it has been shown that intensive treatment with insulin may adversely affect the development of retinopathy. In the DCCT, early worsening of retinopathy was observed in 13.1% of 711 patients assigned to intensive treatment compared to 7.6% of 728 patients assigned to conventional treatment at the 6- and/or 12-month visit. The Diabetes Control and Complications Trial Research Group. “Early worsening of diabetic retinopathy in the Diabetes Control and Complications Trial.” Arch. Ophthalmol. 116:874-886 (1998). Higher HbA_{1c} levels at screening and reduction of this level during the first 6 months after randomization were therefore the most important risk factor for early worsening. Maberley, et al. conducted a study of 157 patients with both Type 1 and 2 diabetes. Insulin treatment was shown to be associated with an increased risk of retinopathy when compared to individuals on dietary therapy alone. Maberley D A L, King W, Cruess A F, et al.; “Risk factors for diabetic retinopathy in the Cree of James Bay.” Ophthalmic Epidemiology 9:153-167 (2002). Similarly, Henricsson, et al. conducted a study in over 1,000 patients with Type 2 diabetes. A change of treatment from oral drugs to insulin was associated with a 100% increased risk of retinopathy progression and a 3-fold increased risk of blindness/visual impairment. Henricsson M, Nilsson A, Janzon L, et al.; “The effect of glycaemic control and the introduction of insulin therapy on retinopathy in non-insulin-dependent diabetes mellitus.” Diabet. Med. 14:123-131 (1997).

If diabetic retinopathy is not well managed, it can have a devastating impact on quality of life. Diabetic retinopathy can lead to visual impairment, which is associated with poor survival. Rajala, et al. reported that the 4-year mortality of people with diabetes was 5-fold compared with age- and sex-matched non-diabetic controls. In their study, excess mortality was attributed mainly to cardiovascular diseases. Rajala U, Pajunpaa H, Koskela P, et al.; “High cardiovascular disease mortality in subjects with visual impairment caused by diabetic retinopathy.” Diabetes Care 23:957-961 (2000). Khaleeli, et al. also reported a significantly higher death rate among those patients with typical retinopathy (26%) than among those without (9%). Congestive cardiac failure and stroke were cited as the main causes of death. Khaleeli A A, fear S, Maitland H, et al.; “Diabetic retinopathy. Outcome at five-year follow-up of 203 people with diabetes. 2: Analysis.” Practical Diabetes International 16:68-70 (1999). Henricsson, et al. conducted a follow-up study of 3,220 patients with diabetes to assess retinopathy and change of treatment to insulin therapy as risk factors for mortality. Death occurred in 263 patients during the mean follow-up time of 3.4 years. Over half (56.3%) of deaths were from cardiovascular disease. It was reported that severe retinopathy, use of antihypertensive drugs and poor glycaemic control predicted death from cardiovascular disease in these patients. Henricsson M, Nilsson A, Heijl A, et al.; “Mortality in diabetic patients participating in an ophthalmological control and screening programme.” Diabet. Med. 14:576-583 (1997).

Diabetic nephropathy is one of the most serious complications of diabetes and the leading cause of end-stage renal disease. Brenner B M, Cooper M E, de Zeeuw D, et al.; “Effects of losartan on renal and cardiovascular outcomes in patients with Type 2 diabetes and nephropathy.” N. Engl. J. Med. 345:861-869 (2001). This microvascular complication is first manifested as an increase in urinary albumin excretion (microalbuminuria), which progresses to overt albuminuria and then to renal failure. Mogensen C E, Christensen C K, Vittinghus E.; “The stages in diabetic renal disease: with emphasis on the stage of incipient diabetic nephropathy.” Diabetes 32(Suppl. 2):64-78 (1983). Although the risk of diabetic nephropathy appears to be similar in Type 1 and Type 2 diabetes, the occurrence of this complication in Type 2 diabetes is a much larger burden in society. Krolewski A S, Warram J H.; “Natural history of diabetic nephropathy: How much can it be changed?” Diabetes Reviews 3:446-459 (1995). A number of factors may interplay in the pathogenesis of diabetic nephropathy, including metabolic, hemodynamic and as yet, poorly defined genetic and/or environmental determinants. The Diabetes Control and Complications Trial Research Group. “Effect of intensive therapy on the development and progression of diabetic nephropathy in the Diabetes Control and Complications Trial.” Kidney Int. 47(6):1703-1720 (1995). Microalbuminuria develops early in the course of nephropathy and is currently considered as an indicator of renal endothelial dysfunction, as well as an independent predictor of cardiovascular risk in individuals with or without diabetes. Gerstein H C, Mann J F, Yi Q et al. HOPE Study Investigators. “Albuminuria and risk of cardiovascular events, death, and heart failure in diabetic and nondiabetic individuals.” JAMA 286(4):421-426 (2001); Waeber B, Feihl F, Ruilope L.; “Diabetes and hypertension.” Blood Press 10(5-6):311-321 (2001).

The prevalence of nephropathy varies dependent on the study population. Data from the UKPDS demonstrated that approximately 25% of patients with Type 2 diabetes develop microalbuminuria or worse nephropathy by 10 years, and it is estimated that almost 50% of patients who develop microalbuminuria do so within 19 years from diagnosis of diabetes. Adler A I, Stevens R J, Manley S E et al, on behalf of the UKPDS Group. “Development and progression of nephropathy in type 2 diabetes: The United Kingdom Prospective Diabetes Study (UKPDS 64).” Kidney Int. 263:225-232 (2003). From any stage of nephropathy, the rate of deterioration to the next stage is 2 to 3% per year.

In comparison to the general population, a disproportionately large percentage of patients with end-stage renal disease have diabetes. The incidence of end-stage renal disease in patients with Type 2 diabetes in particular, is rising sharply in many regions of the world. Data from a prospective cohort study by Brancati, et al. confirm that diabetes mellitus is a strong independent risk factor for ESRD, even for ESRD ascribed to causes other than diabetes, including hypertension. Brancati F L, Whelton P K, Randall B L, et al.; “Risk of end-stage renal disease in diabetes mellitus: A prospective cohort study of men screened for MRFIT.” JAMA 278(23):2069-2074 (1997). A study by Klein, et al. reported that the 10-year incidence of renal insufficiency and failure in people with Type 1 diabetes was high (14.4%), and varied from 5.6% in those with 0-9 years of diabetes to 33.5% in those with ≧35 years of diabetes. Klein R, Klein B E K, Moss S E, et al.; “The 10-year incidence of renal insufficiency in people with type 1 diabetes.” Diabetes Care 22:743-751 (1999).

The main risk factors for the frequency, severity and progression of nephropathy include hyperglycaemia, hypertension, duration of diabetes, age of onset, protein overload and smoking. There is also evidence to suggest that some individuals with diabetes have a genetic predisposition to diabetic nephropathy. The level of glycaemic control appears to be the dominant risk factor for the occurrence of microalbuminuria, whereas progression through the more advanced stages of nephropathy is affected by hypertension, hypercholesterolaemia and genetic factors.

To examine the relation between the degree and duration of hyperglycaemia and the prevalence of microalbuminuria, Krolewski, et al. conducted a study in a large cohort (n=1,613) of patients with Type 1 diabetes. The prevalence of microalbuminuria increased with increasing postpubertal duration of diabetes and, within each six-year interval of disease duration, it showed an overall trend towards increasing with the HbA₁ value (Table 3). Krolewski A S, Laffel L M B, Krolewski M, et al.; “Glycosylated hemoglobin and the risk of microalbuminuria in patients with insulin-dependent diabetes mellitus.” N. Eng. J. Med. 332:1251-1255 (1995).

TABLE 3
Odds ratios for the effect of variations in HbA₁ values
on the development of microalbuminuria

| Dura- | | | | | |
|---|---|---|---|---|---|
| tion | | | | | |
| of | Haemoglobin A₁ values in 1990-1991 | | | | |
| dia- | 5.9-8.8% | 8.9-9.8% | 9.9-10.7% | 10.8-11.9% | 12.0-21.3% |
| betes | Odds ratio (total no. of patients) | | | | |
| 1-6 | 1.0 (104)* | 1.6 (65) | 2.6 (64) | 2.2 (49) | 5.8 (74) |
| 7-12 | 2.4 (58) | 2.3 (82) | 2.4 (81) | 6.8 (94) | 13.2 (107) |
| 13-18 | 2.3 (47) | 4.7 (69) | 3.9 (52) | 7.5 (52) | 28.8 (43) |
| 19-24 | 11.3 (45) | 15.0 (40) | 14.3 (44) | 12.1 (52) | 23.6 (35) |
| 25-32 | 7.1 (27) | 7.9 (25) | 13.0 (35) | 19.0 (37) | 12.5 (21) |

*The prevalence of microalbuminuria was 3.8% in the reference group (patients with the lowest HbA₁ values [range, 5.9-7.9%; mean 7.3%])

Randomised intervention trials have shown that intensive treatment delays the onset and slows the progression not only of retinopathy, but also nephropathy in patients with Type 1 diabetes. The Diabetes Control and Complications Trial Research Group. “The effect of intensive treatment of diabetes on the development and progression of long-term complications in insulin-dependent diabetes mellitus.” N. Eng. J. Med. 329:977-986 (1993). In the DCCT intensive therapy reduced the mean adjusted risk of the cumulative incidence of microalbuminuria in the primary prevention cohort by 34%, and the albumin excretion rate by 15% after the first year of therapy. In the secondary prevention cohort, intensive therapy reduced the mean adjusted risk of microalbuminuria by 43%, the risk of a more advanced level of microalbuminuria by 56%, and the risk of clinical albuminuria by 56%. The Diabetes Control and Complications Trial Research Group. “Effect of intensive therapy on the development and progression of diabetic nephropathy in the Diabetes Control and Complications Trial.” Kidney Int. 47(6):1703-1720 (1995). In the initial 4 years of EDIC very few patients in the intensive treatment group progressed to proteinuria or renal insufficiency. The Writing Team for the Diabetes Control and Complications Trial/Epidemiology of Diabetes Interventions and Complications Research Group. “Effect of intensive therapy on the microvascular complications of Type 1 diabetes mellitus.” JAMA 287:2563-2569 (2002). Yearly incidence of nephropathy begins to rise at 10 years duration of Type 1 diabetes and reaches a peak between 10 and 15 years. Epidemiology of Diabetes Interventions and Complications (EDIC) Research Group. “Epidemiology of Diabetes Interventions and Complications (EDIC).” Diabetes Care 22:99-111 (1999).

Hypertension is an important risk factor for microalbuminuria. The Hypertension in Diabetes Study reported that hypertensive patients suffered a higher prevalence of microalbuminuria compared with normotensive ones (24% versus 14%). Intensive blood pressure control in normotensive patients with Type 2 diabetes slows progression to incipient and overt diabetic nephropathy. The Hypertension in Diabetes Study Group. “Hypertension in Diabetes Study (HDS): 1. Prevalence of hypertension in newly presenting type 2 diabetic patients and the association with risk factors for cardiovascular and diabetic complications.” J. Hypertens 11(3):309-317 (1993). The UKPDS reported that, at 6 years of follow-up, a smaller proportion of patients assigned to tight control of blood pressure, aiming for a blood pressure <150/85 mm Hg, had a urinary albumin concentration of ≧50 mg/l, a 29% reduction in risk. Turner R, Holman R, Stratton I, et al.; “Tight blood pressure control and risk of macrovascular and microvascular complications in type 2 diabetes: UKPDS 38.”BMJ 317:703-713 (1998). Schrier, et al. also reported that intensive blood pressure control slows the progression to incipient and overt diabetic nephropathy and decreases the progression of diabetic retinopathy in normotensive (BP <140/90 mmHg) Type 2 diabetic patients. Schrier R W, Estacio R O, Esler A, et al.; “Effects of aggressive blood pressure control in normotensive type 2 diabetic patients on albuminuria, retinopathy and strokes.” Kidney Int. 61:1086-1097 (2002). Over a 5-year period, a lower percentage of patients in the intensive group progressed from normoalbuminuria to microalbuminuria and microalbuminuria to overt albuminuria.

Evidence of a link between development and progression of nephropathy and duration of diabetes can be found in a number of studies. The UKPDS showed that, at diagnosis, 92.7% of patients with Type 2 diabetes had no nephropathy, 7.3% had microalbuminuria or worse nephropathy, and 0.7% had macroalbuminuria or worse nephropathy. Adler A I, Stevens R J, Manley S E et al, on behalf of the UKPDS Group. “Development and progression of nephropathy in type 2 diabetes: The United Kingdom Prospective Diabetes Study (UKPDS 64).” Kidney Int. 63:225-232 (2003). Patients were followed for a median 10.4 years. Following diagnosis, progression to microalbuminuria occurred at 2.0% per year, from microalbuminuria to macroalbuminuria at 2.8% per year, and from macroalbuminuria to elevated plasma creatinine or renal replacement therapy at 2.3% per year. Ten years following diagnosis, microalbuminuria or worse nephropathy was present in 24.9% of patients; macroalbuminuria or worse nephropathy in 5.3% of patients and elevated plasma creatinine or renal replacement therapy in 0.8% of patients (Table 6).

TABLE 4
Prevalence of nephropathy over 15 years

| | | | | Elevated plasma |
|---|---|---|---|---|
| | | Microalbuminuria | Macroalbuminuria | creatinine or renal |
| | | or worse | or worse | replacement |
| | Number | nephropathy | nephropathy | therapy |
| Time | alive and | Observed % | Observed % | Observed % |
| (years) | examined | (95% CI) (n) | (95% CI) (n) | (95% CI) (n) |
| 0 | 5097 | 7.3% (6.6-8.0) (370) | 0.7% (0.5-1.0) | 0% (0.0-0.0) (0) |
| | | | (37) | |
| 5 | 4791 | 17.3% (16.3-18.4) | 3.1% (2.6-3.6) | 0.4% (0.2-0.6) |
| | | (830) | (149) | (19) |
| 10 | 2799 | 24.9% (23.3-26.5) | 5.3% (4.5-6.1) | 0.8% (0.5-1.1) |
| | | (696) | (148) | (22) |
| 15 | 435 | 28.0% 23.8-32.3) | 7.1% (4.7-9.5) | 2.3% (0.9-3.7) |
| | | (122) | (31) | (10) |

The risk of developing nephropathy has been shown to be similar in Type 1 and Type 2 diabetes. In a study by Hasslacher, et al. the cumulative risk of proteinuria occurring after 20 years was 27% in Type 2 diabetes and 28% in Type 1 diabetes. After 25 years this increased to 57% in Type 2 diabetes and 46% in Type 1 diabetes. The cumulative risk of renal failure (serum creatinine >1.4mg/dl) after 3 years of persisting proteinuria was found to be 41% in both Type 1 and 2 diabetes. After 5 years it was found to be 63% in Type 2 and 59% in Type 1 diabetes. Hasslacher Ch, Ritz E, Wahl P, et al.; “Similar risks of nephropathy in patients with type I or type II diabetes mellitus.” Nephrology Dialysis Transplantation 4:859-863 (1989).

The Pittsburgh Epidemiology of Diabetes Complications Study of 657 patients with Type 1 diabetes with a mean duration of 20 years found no significant differences for microalbuminuria. Overt nephropathy was marginally more prevalent in patients aged 30 years or over with diabetes duration of 15-19 years and less prevalent in patients aged 30 years or over with diabetes duration of 25-29 years (Table 7). Orchard T J, Dorman J S, Maser R E, et al.; “Prevalence of complications in IDDM by sex and duration. Pittsburgh Epidemiology of Diabetes Complications Study II.” Diabetes 39:1116-1124 (1990).

TABLE 5
Percentage of patients with two stages of nephropathy over time

| Duration | Aged 18-29 years | | | Aged ≧ 30 years | | |
|---|---|---|---|---|---|---|
| of | | | Overt | | | Overt |
| diabetes | | Microalbuminuria | nephropathy | | Microalbuminuria | nephropathy |
| (years) | n | (%) | (%) | n | (%) | (%) |
| 5-9 | 45 | 13 | 2 | | | |
| 10-14 | 106 | 18 | 12 | 1 | 0 | 0 |
| 15-19 | 120 | 23 | 23 | 30 | 13 | 40 |
| 20-24 | 50 | 26 | 26 | 65 | 22 | 39 |
| 25-29 | 13 | 8 | 62 | 93 | 24 | 28 |
| 30+ | | | | 69 | 29 | 44 |

Several studies have shown the relationship between smoking and the development of nephropathy in diabetes. Chase, et al. conducted a study of 359 patients with Type 1 diabetes. When compared with non-smokers, smokers were at 2.8 greater risk for albuminuria. Chase H P, Garg S K, Marshall G, et al.; “Cigarette smoking increases the risk of albuminuria among subjects with type 1 diabetes.” JAMA 265:614-617 (1991). Sawicki, et al. also found that cigarette smoking represents an important factor associated with the progression of nephropathy in treated hypertensive patients with Type 1 diabetes. Progression of nephropathy was less common in non-smokers (11%) than in smokers (53%), and ex-smokers (33%). Sawicki P T, Didjurgeit U, Muhlhauser I, et al. “Smoking is associated with progression of diabetic nephropathy.” Diabetes Care 17:126-131 (1994). Rossing, et al. also reported smoking to be associated with progression in albuminuria, which is similar to the findings of the EURODIAB IDDM Complications Study. Rossing P, Hougaard P, Parving H H. “Risk factors for development of incipient and overt diabetic nephropathy in type 1 diabetic patients: a 10-year prospective observational study.” Diabetes Care 25(5):859-864 (2002).

Age at onset of diabetes is a documented risk factor for the development and progression of nephropathy. The role of puberty in the prevalence and correlations of microvascular complications has been evaluated in a number of studies. Kostraba, et al. evaluated the contribution of diabetes duration both pre- and post-puberty to the development of retinopathy and nephropathy. The prevalence of overt nephropathy in 522 adult patients with Type 1 diabetes (mean duration 20.8 years) was significantly greater in patients diagnosed during puberty compared with those diagnosed before puberty. When nephropathy was evaluated by postpubertal duration, the prevalence in the pubertal-onset group was similar to that in the pre-pubertal-onset group. Kostraba J N, Dorman J S, Orchard T J, et al.; “Contribution of diabetes duration before puberty to development of microvascular complications in IDDM subjects.” Diabetes Care 12:686-693 (1989).

Serum AGE levels also seem to play a role in the progression of nephropathy and have been found localised in nodular lesions on nephropathic kidneys, impairing the assembly of proteins in vivo. Makino H, Shikata K, Kushiro M, et al.; “Roles of advanced glycation end-products in the progression of diabetic nephropathy.” Nephrol. Dial. Transplant 11 (Suppl. 5):76-80 (1996). The accumulation of AGEs at these lesions is in itself determined by many factors including renal function, glycaemic control, age of the patient and renal tissue damage in patients with diabetic nephropathy. Sugiyama S, Miyata T, Horie K, et al.; “Advanced glycation end-products in diabetic nephropathy.” Nephrol. Dial. Transplant 11:91-94 (1996).

Nephropathy is currently a major health problem, which will rise in the future as the incidence of diabetes increases and the age of onset declines. Koulouridis observed that nephropathy progresses from microalbuminuria and proteinuria to ESRD over 10 to 20 years. Koulouridis E.; “Diabetic nephropathy in children and adolescents and its consequences in adults.” Journal of Pediatric Endocrinology & Metabolism 14:1367-1377 (2001). Once in the advanced stages of diabetic nephropathy, patients are at high risk of cardiovascular death as well as renal failure.

In the UKPDS, the reported annual death rates were 1.4% in patients at the “no nephropathy stage”; 3.0% at the “microalbuminuria” stage, 4.6% at the “macroalbuminuria” stage, and 19.2% in patients with an “elevated plasma creatinine or renal replacement therapy”. Patients with elevated plasma creatinine but without renal replacement therapy had an annual death rate of 18.9%. Death was usually due to cardiovascular disease. At 10 years, decreasing proportions of patients were alive for worsening stages of nephropathy. Adler A I, Stevens R J, Manley S E et al, on behalf of the UKPDS Group. “Development and progression of nephropathy in type 2 diabetes: The United Kingdom Prospective Diabetes Study (UKPDS 64).” Kidney Int. 63:225-232 (2003).

Genetic factors may also play a role in the development of nephropathy. Tarnow, et al. conducted a study on parents of Type 1 diabetic patients with nephropathy. It was found that there was an increased early mortality rate from cardiovascular disease in these parents. It was suggested that the genetic risk factor thought to be responsible for a predisposition to cardiovascular disease. The ACE/ID polymorphism may be involved, possibly leading to increased angiotensin I-converting enzyme (ACE) levels in the patients with nephropathy and their parents, and increasing the risk of nephropathy.

Tarnow L, Rossing P, Nielsen F S, et al.; “Cardiovascular morbidity and early mortality cluster in parents of type 1 diabetic patients with diabetic nephropathy.” Diabetes Care 23:30-33 (2000).

Neuropathy is also very common and often the most difficult complication to treat, because it is often asymptomatic in patients with diabetes. It remains untreatable except by palliative measures. Diabetic neuropathy has diverse manifestations affecting both the somatic and autonomic nervous systems. Distal symmetric sensorimotor polyneuropathy is the most common form, and is the leading cause of lower limb amputation. Most people with distal symmetric sensorimotor polyneuropathy are asymptomatic or mildly symptomatic, and the syndrome may only be detected with careful physical examination. In addition to the symmetric polyneuropathies, people with diabetes are also susceptible to a variety of asymmetric or focal peripheral neuropathies (Table 6). Greene D A, Stevens M J, Feldman E L.; “Diabetic neuropathy: scope of the syndrome.” Am. J. Med. 107:2S-8S (1999).

TABLE 6
Classification of diabetic neuropathy

Diffuse
Distal symmetric sensorimotor polyneuropathy
Autonomic neuropathy (sudomotor, cardiovascular, gastrointestinal,
genitourinary)
Symmetric proximal lower limb motor neuropathy (amyotrophy)
Focal
Cranial neuropathy
Radiculopathy/plexopathy
Entrapment neuropathy
Asymmetric lower limb motor neuropathy (amotrophy)

The individual with diabetic neuropathy may present with a wide variety of symptoms and signs associated with different syndromes depending on which nerves are affected and the degree of impairment. Several symptom scores have been developed to assess symptoms of diabetic neuropathy. Cohen, et al. based the presence and staging of peripheral neuropathy on neurological symptom score (NSS), neurological disability score (NDS), autonomic function testing (AFT) and quantitative sensory examination (QSE). Cohen J A, Barret W J, Faldut D, et al.; “Risks for sensorimotor peripheral neuropathy and autonomic neuropathy in non-insulin dependent diabetes mellitus (NIDDM).” Muscle & Nerve 21:72-80 (1998). Shalitin, et al. used a bedside scoring method for diabetic peripheral neuropathy. Shalitin S, Josefsberg Z, Lilos P, et al.; “Bedside scoring procedure for the diagnosis of diabetic peripheral neuropathy in young patients with type 1 diabetes mellitus.” Journal of Pediatric Endocrinology & Metabolism 15:613-620 (2002). Neuropathy can also be assessed according to composite scores and neurological examinations. Litchy W, Dyck P, Tesfaye S, et al. The MBBQ study group.; “Diabetic peripheral neuropathy (DPN) assessed by neurological examination (NE) and composite scores (CS) is improved with LY333531 treatment.” Diabetes 51:A197-A198 (2002). The vibration perception threshold (VPT) score is a useful predictor of neuropathy in diabetic patients. Coppini D V, Weng C, Young P J, et al.; “The ‘VPTscore’—a useful predictor of neuropathy in diabetic patients. [letter]”Diabet. Med. 17:488-490 (2000). A VPT score of more than 10.1 can identify diabetic patients at risk of developing peripheral neuropathy. Coppini D V, Wellmer A, Weng C, et al.; “The natural history of diabetic peripheral neuropathy determined by a 12 year prospective study using vibration perception thresholds.” Journal of Clinical Neuroscience 8:520-524 (2001). Age, disease duration, skin changes in feet and myocardial infarction/ischaemia are all associated factors and can help identify patients at risk.

The prevalence of neuropathy in diabetic populations varies considerably, due to the variation in criteria for diagnosis, patient selection and employment of different diagnostic tests. O'Hare J A, Abuaisha F, Geoghegan M.; “Prevalence and forms of neuropathic morbidity in 800 diabetics.” Irish Journal of Medical Science 163:132-135 (1994); Tesfaye S, Stevens L K, Stephenson J M, et al.; “Prevalence of diabetic peripheral neuropathy and its relation to glycaemic control and potential risk factors: the EURODIAB IDDM Complications Study.” Diabetologia 39(11):1377-1384 (1996). In the EURODIAB IDDM Complications Study, 28% of patients with Type 1 diabetes had diabetic neuropathy. Fedele, et al. reported a similar prevalence rate of diabetic neuropathy (32.3%) in patients with Type 1 and Type 2 diabetes. Fedele D, Comi G, Coscelli C, et al.; “A multicentre study on the prevalence of diabetic neuropathy in Italy.” Diabetes Care 20:836-843 (1997). In addition, a total of 2,033 patients with diabetic neuropathy also underwent a quantitative neurological examination and formal nerve conduction studies and were classified into one of five groups: 16.5% had no neuropathy, 19.4% had borderline neuropathy, 22.3% had mild neuropathy, 29.1% had moderate neuropathy and 12.7% had severe neuropathy. Of the patients with neuropathy, 64.1% were aged between 58 and 59 years with disease duration between 12.4+/−8.4 years (mild neuropathy) and 15.6+/−9.7 years (severe neuropathy). High prevalence rates of diabetic neuropathy were reported in the Pittsburgh Epidemiology of Diabetes Complications Study and the DCCT, which used similar criteria to Fedele, et al. Maser R E, Steenkiste A R, Dorman J S, et al.; “Epidemiological correlates of diabetic neuropathy. Report from the Pittsburgh Epidemiology of Diabetes Complications Study.” Diabetes 38:1456-1461 (1989); The Diabetes Control and Complications Trial Research Group. “Factors in development of diabetic neuropathy. Baseline analysis of neuropathy in feasibility phase of Diabetes Control and Complications Trial (DCCT).” Diabetes 37:476-481 (1988). In the Seattle Diabetic Foot Study, 50% of the study participants for whom neuropathy testing was available were found to have peripheral sensory neuropathy at baseline. Adler A I, Boyko E J, Ahroni J H, et al.; “Risk factors for diabetic peripheral sensory neuropathy: results of the Seattle Prospective Diabetic Foot Study.” Diabetes Care 20:1162-1167 (1997).

Different forms of neuropathy can co-exist; however, peripheral neuropathy does not always co-exist with autonomic neuropathy. Tentolouris, et al. reported a similar prevalence of pure autonomic neuropathy in Type 1 diabetes (28.8%) and Type 2 diabetes (20.7%). Fewer patients with Type 1 diabetes (13.5%) however, were found to have pure peripheral neuropathy compared with patients with Type 2 diabetes (20.7%). Similarly, fewer patients with Type 1 diabetes (28.8%) were found to have both peripheral and autonomic neuropathy compared with patients with Type 2 diabetes (45.3%). Tentolouris N, Pagoni S, Tzonou A, et al.; “Peripheral neuropathy does not invariably co-exist with autonomic neuropathy in diabetes mellitus.” European Journal of Internal Medicine 12:20-7 (2001). Barbosa, et al. conducted a study in 93 patients with Type 2 diabetes and found that 80% had symptoms of polyneuropathy; however, distal symmetrical polyneuropathy was only present in 32.2%. Barbosa A P, Medina J L, Ramos E P, et al.; “Prevalence and risk factors of clinical diabetic polyneuropathy in a Portuguese primary health care population.” Diabetes & Metabolism 27:496-502 (2001).

Compared to the wealth of information on the risk factors affecting retinopathy and nephropathy, data regarding neuropathy are far less exhaustive, possibly due to greater difficulties diagnosing and classifying it. Available evidence implicates metabolic control, age, duration of diabetes, the presence of retinopathy, cigarette smoking, and height. In the EURODIAB IDDM Complications Study significant correlations were observed between the presence of diabetic peripheral neuropathy and increasing age, duration of diabetes, HbA_{1c}, height, the presence of background or proliferative diabetic retinopathy, smoking, high-density lipoprotein cholesterol and the presence of cardiovascular disease. In addition, new associations were identified, namely with elevated diastolic blood pressure, the presence of severe ketoacidosis, an increase in the levels of fasting triglyceride, and the presence of microalbuminuria. Tesfaye S, Stevens L K, Stephenson J M, et al.; “Prevalence of diabetic peripheral neuropathy and its relation to glycaemic control and potential risk factors: the EURODIAB IDDM Complications Study.” Diabetologia 39(11):1377-1384 (1996). The Seattle Prospective Diabetic Foot Study also identified numerous clinical and historical variables associated with an increased risk of diabetic peripheral sensory neuropathy, including age at entry into the study, glycohaemoglobin levels, history of lower-extremity ulceration, and body height, as patients who developed neuropathy during the 10-year follow up period were more likely to be taller compared to those who remained free of neuropathy. Adler A I, Boyko E J, Ahroni J H, et al.; “Risk factors for diabetic peripheral sensory neuropathy: results of the Seattle Prospective Diabetic Foot Study.” Diabetes Care 20:1162-1167 (1997).

Dickinson, et al. found that hyperglycaemic-induced oxidative stress may affect the development of neuropathy as it can result in decreased nerve conduction velocity. Dickinson P J, et al. “Neurovascular disease, antioxidants and glycation in diabetes.” Diabetes. Metab. Res. Rev. 18:260-272 (2002). Mustonen, et al. demonstrated that over a 4-year period, the deterioration of autonomic nervous function score in patients with Type 1 and Type 2 diabetes is associated with poor glycaemic control at baseline. Autonomic nervous function score increased in patients with Type 2, but did not change in patients with Type 1 or in control subjects. Mustonen J, et al.; “Changes in autonomic nervous function during the 4 year follow-up in middle-aged diabetic and nondiabetic subjects initially free of coronary heart disease.” Journal of Internal Medicine 241:227-235 (1997). Using Cox proportional hazards model, a study by Forrest, et al., showed that glycaemic control and hypertension were independent predictors of distal symmetric polyneuropathy in patients with Type 1 diabetes. Forrest K Y, et al.; “Hypertension as a risk factor for diabetic neuropathy: a prospective study.” Diabetes 46:665-670 (1997).

The DCCT demonstrated that intensive therapy with three or more daily insulin injections or continuous subcutaneous insulin infusion reduced the development of confirmed clinical neuropathy by 64% in the combined cohorts after 5 years of follow-up compared with conventional therapy. The prevalence of abnormal nerve conduction and abnormal autonomic nervous system function were also reduced by 44% and 53%, respectively. Further data from the DCCT confirm that the electrophysiological abnormalities associated with diabetic neuropathy are delayed or prevented by intensive diabetes treatment.

As with diabetic retinopathy and nephropathy, duration of diabetes is a documented risk factor for the development of diabetic neuropathy. Toyry, et al. reported that the frequency of different subtypes of neuropathy increases over time in patients with Type 2 diabetes when evaluated at five and ten years (Table 7). Toyry J P, et al.; “Occurrence, predictors, and clinical significance of autonomic neuropathy in NIDDM: Ten year follow-up from the diagnosis.” Diabetes 45:308-315 (1996). Partenen, et al. also reported an increased prevalence of polyneuropathy among patients with Type 2 diabetes with time. Baseline prevalence of definite or probable neuropathy was 8.3% as compared with 2.1% among control subjects. After 10 years, these values increased to 41.9% and 5.8%, respectively. Partanen J, et al.; “Natural history of peripheral neuropathy in patients with non insulin-dependent diabetes mellitus.” N. Engl. J. Med. 333:89-94 (1995).

TABLE 7
Prevalence of sympathetic and parasympathetic
neuropathy over 10 years

| | | | Combined |
|---|---|---|---|
| | Parasympathetic | Sympathetic | autonomic |
| | neuropathy | neuropathy | neuropathy (both) |
| After 5 years | 19.6% | 6.8% | 2.1% |
| After 10 years | 65.0% | 24.4% | 15.2% |

Evidence shows that cigarette smoking is also associated with the development of various types of diabetic neuropathy in patients with Type 1 and Type 2 diabetes. Mitchell, et al. reported that patients with Type 1 diabetes who were current or ex-smokers were significantly more likely to have neuropathy than those who had never smoked. In addition, the prevalence of neuropathy increased with increased number of pack-years smoked. Mitchell B D, et al.; “Cigarette smoking and neuropathy in diabetic patients.” Diabetes Care 13:434-437 (1990). Sands, et al. conducted a prospective study of 231 people with Type 2 diabetes who were free of distal sensory neuropathy at baseline for an average of 4.7 years. The adjusted incidence rate of smokers was 2.2 times greater than for non-smokers. Sands M L, et al.; “Incidence of distal symmetric (sensory) neuropathy in NIDDM. The San Luis Valley Diabetes Study.” Diabetes Care 20:322-329 (1997). Significant correlation between the presence of diabetic peripheral neuropathy and cigarette smoking has also been reported in the EURODIAB IDDM Prospective Complications Study. Tesfaye S, et al.; “Prevalence of diabetic peripheral neuropathy and its relation to glycaemic control and potential risk factors: the EURODIAB IDDM Complications Study.” Diabetologia 39(11):1377-1384 (1996).

People with diabetes are up to 15 times more likely to have a lower limb amputation than non-diabetic individuals, and foot problems are the most common reason for diabetes-related hospitalisation. In a study by O'Hare, et al., 22.9% of the population reported a problem from neuropathy. Symptomatic complications of polyneuropathy were the predominant source of neuropathic morbidity in while mononeuropathy and amyotrophy were found to be rare. No significant difference in the prevalence of neuropathic complications was reported between Type 1 and Type 2 diabetes (Table 8); O'Hare J A, et al., “Prevalence and forms of neuropathic morbidity in 800 diabetics.” Irish Journal of Medical Science 163:132-135 (1994).

TABLE 8
Prevalence of symptoms and complications of diabetic neuropathy

| | Type 1 (%) | Type 2 (%) | All (%) |
|---|---|---|---|
| | (n = 336) | (n = 464) | (n = 800) |
| Pain/paraesthesia | 12.5 | 13.8 | 13.3 |
| Feeling loss | 6.5 | 6.7 | 6.6 |
| Restless legs syndrome | 6.0 | 10.8 | 8.8 |
| Ulcers | 1.5 | 2.2 | 1.9 |
| Amyotrophy | 0.3 | 1.1 | 0.8 |
| Oculomotor | 0.3 | 0 | 0.1 |
| Peroneal | 0 | 0.2 | 0.1 |
| Truncal | 0.3 | 0 | 0.1 |
| Impotence (<65 yrs) | 13 | 26 | 20 |
| Postural hypotension | 0.9 | 0.9 | 0.9 |
| Diarrhoea | 1.5 | 0.6 | 1.0 |
| Any neuropathy | 22.0 | 23.5 | 22.9 |

Consistent with the mortality from retinopathy and nephropathy, deaths in patients with neuropathy are frequently due to cardiovascular disease. Forsblom C M, et al.; “Risk factors for mortality in Type II (non-insulin-dependent) diabetes: evidence of a role for neuropathy and a protective effect of HLA-DR4.”Diabetologica 41:1253-1262 (1998). Most clinical signs and symptoms of autonomic neuropathy however, do not progress, so autonomic function tests must be used to identify patients at risk of the morbidity associated with neuropathy. Levitt N S, et al.; “The natural progression of autonomic neuropathy and autonomic function tests in a cohort of people with IDDM.” Diabetes Care 19:751-754 (1996).

As set forth in this invention, a “diabetes microvascular complications score” or “DMCS” refers to an aggregate score of a patient's overall microvascular activity, i.e. reflecting, for example, at least the patient's retinopathy, neuropathy and nephropathy status. Similarly, a “diabetes microvascular complications scoring tool” or “DMCS tool” refers to medical diagnostic tool comprising a template and a risk assessment standard, which can refer to any data in the form of a chart, table, database, or combinations thereof that can be used or accessed by a patient, attending physician, or medical provider that classifies diabetic complications in terms of prognosis or risk factors and in terms of the inter-relationship among said diabetic complications. The DMCS tool assigns specific numerical values to reflect the severity of each particular diabetic complication to be measured. At least three different diabetic complications are measured and then added to provide a composite score. Once a pre-determined minimum composite score is reached, it becomes appropriate for a patient to receive or a doctor to prescribe an appropriate compound, preferably ruboxistaurin or a pharmaceutically acceptable salt thereof, to treat the diabetic complications.

For example, the template can be a table that assigns a value for each complication measured, e.g., the higher the number, the greater the severity of a given complication. The patient, physician, or medical provider then assesses the level of severity of each complication, records the value provided by the tool for each complication and adds the values to attain a DMCS. The DMCS is then measured against the risk assessment standard to determine whether the level of risk warrants treatment with an appropriate drug to treat the diabetic complications. Prospective examples are provided below. In each of the examples, each complication is assessed a particular severity value (0-5), but these numbers are illustrative only. Additional measures may be assessed to determine the severity of each complication and the individual component stages of the complications may vary or have additional or fewer or amended components:

### EXAMPLE 1

First, a standard template is referenced which provides fixed numerical values to the severity of a given diabetic complication based upon pre-determined risk factors as follows:

| Score | Retinopathy | Neuropathy | Nephropathy |
|---|---|---|---|
| 0 | None | Normal | None |
| 1 | Mild | Abnormal nerve conduction | Microalbuminuria |
| | | velocity | |
| 2 | Moderate | Impaired vibration assessment | Proteinuria |
| 3 | Severe | Positive/negative symptoms | End stage renal |
| | | | disease |
| 4 | Proliferative | Foot ulcer | |
| | diabetic | | |
| | retinopathy | | |
| | (PDR) | | |
| 5 | | Amputation | |

Note, these fixed numerical values are arbitrarily given the scores of 0-5 for ease of understanding for now, however, the eventual values may change, reflecting the relative weighting of the individual components of the complications. The scores are then added up to give a total aggregated score, or DMCS, which is then compared to a risk assessment standard of scores which reflect different “risk” categories. The risk assessment standard comprises of different “risk” categories (eg “low”, “medium” or “high”) which are reflective of the aggregate DMCS attained by a patient. There are recommendations on the management of a particular patient depending on the “risk” category a patient's DMCS falls in. Examples of the “risk” categories and their subsequent recommendations could be as follows: that those characterized as “low risk” should maintain regular checks and optimize glycemic/blood pressure (“BP”)/lipid controls. Those characterized as “medium risk” should have more frequent checks, optimize glycemic/blood pressure (“BP”)/lipid controls, and should consider drug treatment. Those characterized as “high risk” should have very frequent checks, optimize glycemic/BP/lipid controls and be recommended drug treatment.

According to this example, the risk assessment standard provides that those patients with a DMCS of 0-3 are characterized as “low risk”; those with a DMCS of 4-8 are “medium risk” and those with a DMCS of 9- 12 are “high risk”. Note, these scores are arbitrary figures only, assigned for simplicity of understanding for now, but they may and probably will vary depending on the relative weighting of the individual components of the complications being assessed. In this example, the patient has proliferative diabetic retinopathy (4), a foot ulcer (4), and proteinuria (2), for a DMCS of ten (10). This DMCS places the patient in the “high risk” category suggesting the physician conduct more frequent checks on said patient than a normal diabetic patient, optimize glycemic/blood pressure/lipid control and to administer an appropriate drug, for example ruboxistaurin or a salt thereof, to treat diabetic microvascular complications.

### EXAMPLE 2

In this example, the patient has moderate diabetic retinopathy (2), abnormal nerve conduction velocity (1), and proteinuria (2), for a composite DMC score of five (5) when measured against the standard template exemplified in Example 1. This aggregate DMC score places the patient in the “medium risk” category suggesting the physician conduct more frequent checks on said patient than a normal diabetic patient, optimize glycemic/blood pressure/lipid control and to consider administering an appropriate drug, for example ruboxistaurin or a salt thereof, to treat diabetic microvascular complications.

The term “pharmaceutically-acceptable salt” as used herein, refers to a salt of a compound of the present invention. It should be recognized that the particular counterion forming a part of any salt of this invention is usually not of a critical nature, so long as the salt as a whole is pharmacologically acceptable and as long as the counterion does not contribute undesired qualities to the salt as a whole.

In general, a compound of the present invention as described herein forms pharmaceutically-acceptable acid addition salts with a wide variety of organic and inorganic acids and include the physiologically-acceptable salts which are often used in pharmaceutical chemistry. Such salts include the pharmaceutically acceptable salts listed in Journal of Pharmaceutical Science, 66, 2-19 (1977), which are known to the skilled artisan. See also, The Handbook of Pharmaceutical Salts; Properties, Selection, and Use. P. H. Stahl and C. G. Wermuth (ED.s), Verlag, Zurich (Switzerland) 2002.

Ruboxistaurin is also known as: (S)-9-((Dimethylamino)methyl)-6,7,10,11-tetrahydro-9H,18H-5,21:12,17-dimethenodibenzo(e,k)pyrrolo(3,4-h)(1,4,13)oxadiaza-cyclohexadecine-18,20(19H)-dione. The mesylate monohydrate of ruboxistaurin is currently in Phase III clinical trials for various microvascular complications to diabetes and is structurally depicted as:

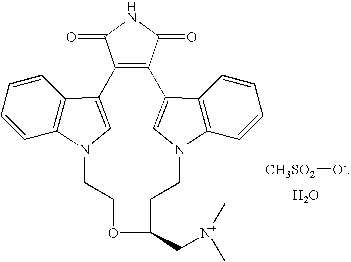

Ruboxistaurin, its pharmaceutically acceptable salts and related compounds are described in Heath, Jr., et al., U.S. Pat. No. 5,552,396. The mesylate salts of ruboxistaurin are specifically described and claimed in U.S. Pat. No. 5,710,145. The synthesis of ruboxistaurin, its salts and related compounds as well as a disclosure that said compounds are useful in the treatment of conditions associated with diabetes mellitus and its complications as well ischemia, inflammation, central nervous system disorders, cardiovascular disease, dermatological disease, Alzheimer's disease and cancer. U.S. Pat. Nos. 5,552,396 and 5,710,145 are hereby incorporated by reference in their entirety as if fully set forth.

Enzastaurin is also known as 1H-pyrrole-2,5-dione, 3-(1-methyl-1H-indol-3-yl)-4-[1-[1-(2-pyridinylmethyl)-4-piperidinyl]-1H-indol-3-yl]-, monohydrochloride; or 3-(1-methyl-1H-indol-3-yl)-4-[1-[1-(pyridin-2-ylmethyl)piperidin-4-yl]-1H-indol-3-yl]-1H-pyrrole-2,5-dione monohydrochloride. The mono-hydrochloride salt of enzastaurin is presently in phase II clinical trials for non-Hodgkins lymphoma, and glioblastoma and is structurally depicted as:

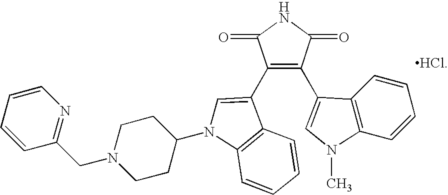

Enzastaurin, its pharmaceutically acceptable salts and related compounds are described in Heath, Jr., et al., U.S. Pat. No. 5,668,152. The mono-hydrochloride salt of enzastaurin is specifically described in PCT Patent Publication No. WO 2004/006928 (Application No. PCT/US2003/019548). The synthesis of enzastaurin, its salts and related compounds as well as a disclosure that said compounds are useful in the treatment of conditions associated with diabetes mellitus and its complications as well ischemia, inflammation, central nervous system disorders, cardiovascular disease, dermatological disease, Alzheimer's disease and cancer. U.S. Pat. No. 5,668,152 and WO 2004/006928 are hereby incorporated by reference in their entirety as if fully set forth.

PKC 412 is a derivative of staurosporin and is also known as N-benzoylstaurosporine; midostaurin; or by its chemical names benzamide, N-[(9S,10R,11R,13R)-2,3,10,11,12,13-hexahydro-10-methoxy-9-methyl-1-oxo-9,13-epoxy-1H,9H-diindolo[1,2,3-gh:3′,2′,1′-lm]pyrrolo[3,4-j][1,7]benzodiazonin-11-yl]-N-methyl-; or N-[(9S,10R,11R,13R)-10-methoxy-9-methyl-1-oxo-2,3,10,11,12,13-hexahydro-9,13-epoxy- 1H,9H-diindolo[1,2,3-gh:3′,2′,1′-lm]pyrrolo[3,4-j][1,7]benzodiazonin-11-yl]-N-methylbenzamide. PKC 412 has the CAS Registry No. 120685-11-2, is currently in clinical trials, is potentially useful in treating various types of cancers and diabetic complications and is structurally depicted as:

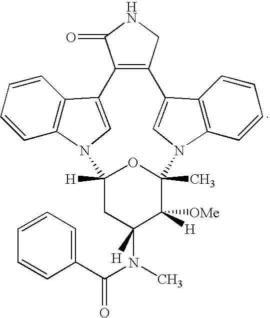

Candesartan cilexetil is also known as (±)-1-hydroxyethyl 2-ethoxy-1-[p-(o-1H-tetrazol-5-ylphenyl)benzyl]-7-benzimidazolecarboxylate, cyclohexyl carbonate (ester). Candesartan cilexetil has the CAS Registry No. 145040-37-5, is currently indicated for the treatment of hypertension in the United States and is structurally depicted as:

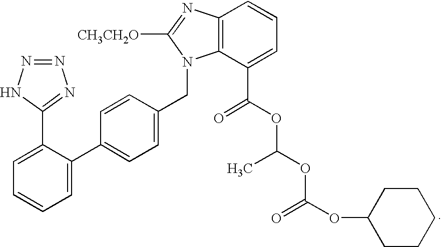

Fidarestat is also known as fidarestatum and by its chemical name (+)-(2S,4S)-6-fluoro-2′,5′-dioxospiro[chroman-4,4′-imidazolidine]-2-carboxamide. Fidarestat has the CAS Registry No. 136087-85-9, is currently in clinical trials as an aldose reductase inhibitor and is structurally depicted as:

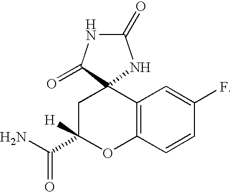

Lidorestat is also known as IDD-676 and by its chemical names 1H-indole-1-acetic acid, 3-[(4,5,7-trifluoro-2-benzothiazolyl)methyl]-, monohydrate and 3-[(4,5,7-triflurobenzothiazol-2-yl)methyl]-1H-indol-1-yl]acetic acid, monohydrate. Lidorestat is currently in clinical trials for the treatment of peripheral diabetic neuropathy, has the CAS Registry No. 245116-90-9 (unhydrated form), and is structurally depicted as:

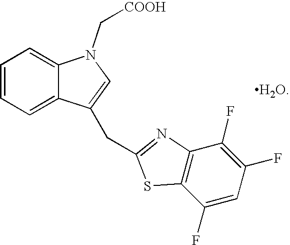

Pyridoxamine is also known as pyridorin and has the CAS Registry No. 524-36-7. The dihydrochloride form is presently being studied as an advanced glycylation end product (AGE) inhibitor. It is structurally depicted as:

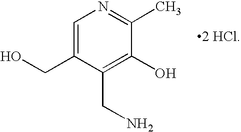

Pegaptanib (see, e.g., www.eyetech.com) is a covalent conjugate of an oligonucleotide of twenty-eight nucleotides in length that terminates in a pentylamino linker, to which two 20-kilodalton monomethoxy polyethylene glycol (PEG) units are covalently attached via the two amino groups on a lysine residue. The sodium salt form of pegaptanib is presently being studied as a selective vascular endothelial growth factor (VEGF) antagonist. Pegaptanib is an aptamer, a pegylated modified oligonucleotide, which adopts a threedimensional conformation that enables it to bind to extracellular VEGF.

Ranibizumab (see, e.g., www.gene.com) is a humanized therapeutic antibody fragment that binds to and inhibits VEGF-A. The VEGF-A protein is believed to play a critical role in angiogenesis and serves as one of the key contributors to physiological or pathological conditions that can stimulate the formation of new blood vessels. The process of angiogenesis is normally regulated throughout development and adult life, and the uncontrolled growth of new blood vessels is an important contributor to a number of pathologic conditions, including wet AMD.

A compound of the invention may be administered in combination, separately, simultaneously or sequentially, with one or more other pharmacologically active agents employed in the management/treatment of diabetic complications particularly agents employed in the treatment/management of associated pain. Suitable other agents thus include:

(i) opioid analgesics, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine and pentazocine;
(ii) nonsteroidal antiinflammatory drugs (NSAIDs), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxicam, sulindac, tolmetin, zomepirac, and their pharmaceutically acceptable salts;
(iii) barbiturate sedatives, e.g. amobarbital, aprobarbital, butabarbital, butabital, mephobarbital, metharbital, methohexital, pentobarbital, phenobartital, secobarbital, talbutal, theamylal, thiopental and their pharmaceutically acceptable salts;
(iv) benzodiazepines having a sedative action, e.g. chlordiazepoxide, clorazepate, diazepam, flurazepam, lorazepam, oxazepam, temazepam, triazolam and their pharmaceutically acceptable salts,
(v) H₁ antagonists having a sedative action, e.g. diphenhydramine, pyrilamine, promethazine, chlorpheniramine, chlorcyclizine and their pharmaceutically acceptable salts;
(vi) miscellaneous sedatives such as glutethimide, meprobamate, methaqualone, dichloralphenazone and their pharmaceutically acceptable salts;
(vii) skeletal muscle relaxants, e.g. baclofen, carisoprodol, chlorzoxazone, cyclobenzaprine, methocarbamol, orphrenadine and their pharmaceutically acceptable salts,
(viii) alpha-2-delta ligands, e.g. gabapentin and pregabalin;
(ix) alpha-adrenergic active compounds, e.g. doxazosin, tamsulosin, clonidine and 4-amino-6,7-dimethoxy-2-(5-methanesulfonamido-1,2,3,4-tetrahydroisoquinol-2-yl)-5-(2-pyridyl)quinazoline;
(x) tricyclic antidepressants, e.g. desipramine, imipramine, amytriptiline and nortriptiline;
(xi) anticonvulsants, e.g. carbamazepine and valproate;
(xii) serotonin reuptake inhibitors, e.g. fluoxetine, paroxetine, citalopram and sertraline;
(xiii) mixed serotonin-noradrenaline reuptake inhibitors, e.g. milnacipran, venlafaxine and duloxetine;
(xiv) noradrenaline reuptake inhibitors, e.g. reboxetine;
(xv) Tachykinin (NK) antagonists, particularly Nk-3, NK-2 and NK-1 antagonists, e.g. (αR,9R)-7-[3,5-bis(trifluoromethyl)benzyl]-8,9,10,11-tetrahydro-9-methyl-5-(4-methylphenyl)-7H-[1,4]diazocino[2,1-g][1,7]naphthridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]methyl]-1,2-dihydro-3H-1,2,4-triazol-3-one (MK-869), lanepitant, dapitant and 3-[[2-methoxy-5-(trifluoromethoxy)phenyl]methylamino]-2-phenyl-piperidine (2S,3S)
(xvi) Muscarinic antagonists, e.g oxybutin, tolterodine, propiverine, tropsium chloride and darifenacin;
(xvii) COX-2 inhibitors, e.g. celecoxib, rofecoxib and valdecoxib;
(xviii) Non-selective COX inhibitors (preferably with GI protection), e.g. nitroflurbiprofen (HCT-1026);
(xix) coal-tar analgesics, in particular, paracetamol;
(xx) neuroleptics, such as droperidol;
(xxi) Vanilloid receptor agonists, e.g. resinferatoxin;
(xxii) Beta-adrenergic compounds such as propranolol;
(xxiii) Local anaesthetics, such as mexiletine;
(xxiv) Corticosteriods, such as dexamethasone
(xxv) serotonin receptor agonists and antagonists;
(xxvi) cholinergic (nicotinic) analgesics; and
(xxvii) miscellaneous analgesic agents, such as Tramadol®.

Thus, the invention further provides a combination comprising a compound of the invention and a compound or class of compounds selected from groups (i)-(xxvii), above. There is also provided a pharmaceutical composition comprising such a combination, together with a pharmaceutically acceptable excipient, diluent or carrier, particularly for the treatment of one or more diabetic microvascular complications.

Combinations of a compound of the present invention and other therapeutic agents may be administered separately, sequentially or simultaneously. Thus, the present invention extends to a kit comprising a compound of the invention, one or more other therapeutic agents, such as those listed above, and a suitable container.

A compound of the present invention may be administered neat (alone) or preferably in the form of a pharmaceutical composition, that is, combined with pharmaceutically acceptable carriers, or excipients, the proportion and nature of which are determined by the solubility and chemical properties of the compound selected, the chosen route of administration, and standard pharmaceutical practice.

As used herein, the term “patient” refers to a warm-blooded animal or mammal which is in need of treating one or more diabetic microvascular complications. It is understood that guinea pigs, dogs, cats, rats, mice, hamsters, and primates, including humans, are examples of patients within the scope of the meaning of the term. Preferred patients include humans.

As used herein, the term “treating” is defined to include its generally accepted meaning which includes preventing, prohibiting, restraining, and slowing, stopping or reversing progression, or severity, and holding in check and/or treating existing characteristics. The present method thus includes both medical therapeutic and/or prophylactic treatment, as appropriate.

As used herein, the term “therapeutically effective amount” means an amount of compound of the present invention which is capable of alleviating the symptoms of the various pathological conditions herein described. The specific dose of a compound administered according to this invention will, of course, be determined by the particular circumstances surrounding the case including, for example, the compound(s) administered, the route of administration, the state of being of the patient, and the pathological condition being treated. A preferred dose range for ruboxistaurin mesylate monohydrate is from 32 mg to about 128 mg, administered once per day.

A compound of the present invention may be administered by a variety of routes. In effecting treatment of a patient afflicted with or at risk of developing the disorders described herein, a compound of the present invention can be administered in any form or mode that makes the compound bioavailable in an effective amount, including oral and parenteral routes. For example, a compound of the present invention may be administered orally, by inhalation, or by the subcutaneous, intramuscular, intravenous, transdermal, intranasal, rectal, occular, topical, sublingual, buccal, or other routes. Oral administration is generally preferred for treatment of the disorders described herein. However, oral administration is not the only preferred route. For example, the intravenous route may be preferred as a matter of convenience or practicality or to avoid potential complications related to oral administration.

One skilled in the art of preparing formulations can readily select the proper form and mode of administration depending upon the particular characteristics of the compound selected, the disorder or condition to be treated, the stage of the disorder or condition, and other relevant circumstances. (Remington's Pharmaceutical Sciences, 18th Edition, Mack Publishing Co. (1990)).

The pharmaceutical compositions may be prepared in a manner well known in the pharmaceutical art. The carrier or excipient may be a solid, semi-solid, or liquid material that can serve as a vehicle or medium for the active ingredient. Suitable carriers or excipients are well known in the art. The pharmaceutical composition may be adapted for oral, inhalation, parenteral, or topical use and may be administered to the patient in the form of tablets, capsules, aerosols, inhalants, suppositories, solutions, suspensions, or the like.

For the purpose of oral therapeutic administration, the compounds may be incorporated with excipients and used in the form of tablets, troches, capsules, elixirs, suspensions, syrups, wafers, chewing gums and the like. These preparations should contain at least 4% of a compound of the present invention, the active ingredient, but may be varied depending upon the particular form and may conveniently be between 4% to about 70% of the weight of the unit. The amount of a compound of the present invention present in compositions is such that a suitable dosage will be obtained.

The tablets, pills, capsules, troches, and the like may also contain one or more of the following adjuvants: binders such as povidone, hydroxypropyl cellulose, microcrystalline cellulose, gum tragacanth or gelatin; excipients such as dicalcium phosphate, starch, or lactose; disintegrating agents such as alginic acid, Primogel, corn starch and the like; lubricants such as talc, hydrogenated vegetable oil, magnesium stearate or Sterotex; glidants such as colloidal silicon dioxide; and sweetening agents, such as sucrose, aspartame, or saccharin, or a flavoring agent, such as peppermint, methyl salicylate or orange flavoring, may be added. When the dosage unit form is a capsule, it may contain, in addition to materials of the above type, a liquid carrier such as polyethylene glycol or a fatty oil. Other dosage unit forms may contain other various materials that modify the physical form of the dosage unit, for example, coatings. Thus, tablets or pills may be coated with sugar, shellac, or other coating agents. Syrups may contain, in addition to the present compounds, sucrose as a sweetening agent and certain preservatives, dyes and colorings and flavors. Materials used in preparing these various compositions should be pharmaceutically pure and non-toxic in the amounts used.

1. A method for treating one or more diabetic microvascular complications in a patient in need of said treatment comprising:
(a) diagnosing the severity of at least three different microvascular complications in said patient by calculating a diabetes microvascular complications score with a diabetes microvascular complications scoring tool; and
(b) administering to said patient in need thereof a therapeutic amount of a compound selected from the group consisting of ruboxistaurin, enzastaurin, PKC 412, candesartan cilexetil, fidarestat, lidorestat, pyridoxamine and pegaptanib, or a pharmaceutically acceptable salt thereof, and ranibizumab; in an amount that is effective in treating one or more diabetic microvascular complications in said patient.

2. The method according to claim 1 wherein said compound is selected from the group consisting of ruboxistaurin, enzastaurin, PKC 412, candesartan cilexetil, fidarestat, lidorestat and pyridoxamine, or a pharmaceutically acceptable salt thereof.

3. The method according to claim 2 wherein said patient is a human.

4. The method according to claim 3 wherein said patient has type I or type II diabetes mellitus.

5. The method according to claim 4 wherein said microvascular complications are selected from retinopathy, neuropathy, and nephropathy.

6. The method according to claim 5 wherein said diabetes microvascular complications scoring tool assesses at least three levels of severity for at least three different diabetic microvascular complications.

7. The method according to claim 6 wherein said compound is ruboxistaurin or a pharmaceutically acceptable salt thereof.

8. The method according to claim 6 wherein said compound is enzastaurin or a pharmaceutically acceptable salt thereof.

9. The method according to claim 6 wherein said compound is PKC 412 or a pharmaceutically acceptable salt thereof.

10. The method according to claim 6 wherein said compound is candesartan cilexetil or a pharmaceutically acceptable salt thereof.

11. The method according to claim 6 wherein said compound is fidarestat or a pharmaceutically acceptable salt thereof.

12. The method according to claim 6 wherein said compound is lidorestat or a pharmaceutically acceptable salt thereof.

13. The method according to claim 6 wherein said compound is pyridoxamine or a pharmaceutically acceptable salt thereof.

14. The method according to any claim 6 wherein said compound is pegaptanib or a pharmaceutically acceptable salt thereof.

15. The method according to claim 6 wherein said compound is ranibizumab.

16. (canceled)

17. (canceled)

18. The method according to claim 7 wherein said compound is administered in an amount of 8, 16, or 32 mg one to three times per day.

19. The method according to claim 18 wherein said compound is ruboxistaurin mesylate.
